(19) United States
(12) Patent Application Publication
(10) Pub. No.: US 2026/0053879 A1
(43) Pub. Date: Feb. 26, 2026

(54) COMPOSITION COMPRISING CURCUMINOIDS, MODIFIED STARCH AND/OR ACACIA GUM AND SAPONINS FOR USE AS A MEDICAMENT

(71) Applicants: Givaudan SA, Vernier (CH); Stichting Wageningen Research, Wageningen (NL)

(72) Inventors: Pascale Elizabeth Renee FANCA-BERTHON, Avignon (FR); Julie LAVAL, Avignon (FR); Nicole DE WIT, Wageningen (NL); Lonneke JANSSEN DUIJGHUIJSEN, Wageningen (NL); Arjan Paul VOS, Wageningen (NL)

(21) Appl. No.: 19/105,391

(22) PCT Filed: Aug. 22, 2022
(86) PCT No.: PCT/EP2022/073358

§ 371 Date: Feb. 21, 2025

(51) Int. Cl. A61K 36/484; A61K 31/12; A61K 31/704; A61K 31/715; A61K 36/21; A61K 36/258; A61K 36/73; A61K 36/82; A61K 36/8945; A61K 36/8965; A61K 36/9066; A61P 1/00; A61P 1/12; A61P 3/06

(52) U.S. Cl. CPC A61K 36/484; A61K 31/12; A61K 31/704; A61K 31/715; A61K 36/21; A61K 36/258; A61K 36/73; A61K 36/82; A61K 36/8945; A61K 36/8965; A61K 36/9066; A61P 1/00; A61P 1/12; A61P 3/06

(57) ABSTRACT

The present invention relates to the use of an improved curcuminoids composition for reducing LPS levels in blood, reducing endotoxemia, protecting the integrity of the intestinal barrier, treating, decreasing and/or preventing leaky gut syndrome, reducing intestinal lipids absorption, reducing ApoB48 levels in blood and/or for in treating, decreasing and/or preventing dyslipidemia in a subject.

### FIELD OF THE INVENTION

The present invention relates to a specific composition comprising curcuminoids, acacia gum and quillaja that has an improved effect in reducing LPS levels in blood, in reducing ApoB48 levels in blood, in reducing endotoxemia, in protecting the integrity of the intestinal barrier and/or in treating, decreasing and/or preventing leaky gut syndrome.

The present invention relates to a specific composition comprising curcuminoids, acacia gum and quillaja that has an improved effect in reducing intestinal lipids absorption, reducing ApoB48 levels in blood and/or in treating, decreasing and/or preventing dyslipidemia.

### BACKGROUND OF THE INVENTION

Turmeric is a widely used spice and also colouring/flavouring substance that is extracted from the rhizomes of a plant that is also named turmeric (Curcuma longa). It has been used for centuries in traditional medicine in Asian countries and has been shown to improve numerous health conditions. Turmeric is on the GRAS (generally recognised as safe) substance list of the US Food and Drugs Administration (FDA).

Turmeric was found to be effective even when given by different routes, including topical, oral or by inhalation, dependent on the intended use. The major constituent of turmeric is curcumin (diferuloylmethane), which constitutes up to 70-85% of the total curcuminoid content, with demethoxycurcumin and bis-demethoxycurcumin comprising the remainder (Aggarwal, B. B., & Shishodia, S. (2004). Annals of the New York Academy of Sciences, 1030, 434-441). Curcumin and curcuminoids have been extensively investigated due to their antioxidant and anti-inflammatory properties, notably regarding their potential efficacy in modulating various health conditions. Turmeric (Curcuma longa) extracts have a long history of use worldwide with a good safety profile. However, very high doses of turmeric standard extracts (up to 6000 mg) or enhanced formulation (600 to 2500 mg) are needed to be administrating to have an effect.

WO02/02138 A1 describes a method for the prevention and/or treatment of endotoxemia associated with inflammatory bowel disease including administering to a mammal in need of such treatment an effective amount of a probiotic.

CN106511353 A describes triterpene compounds in preparation of medicines for inhibiting synthesis and secretion of intestinal ApoB48. In particular, to the application in the preparation of medicaments for resisting hyperlipidemia, cardiovascular and cerebrovascular diseases, atherosclerosis and the like.

Akira Asai et al. describe an effect on the lipid accumulation in rodent's livers using high dosage of curcuminoids (Dietary Curcuminoids Prevent High-Fat Diet-Induced Lipid Accumulation in Rat Liver and Epididymal Adipose Tissue, The Journal of Nutrition, Volume 131, Issue 11, November 2001, Pages 2932-2935).

### SUMMARY OF THE INVENTION

The inventors have surprisingly found that a specific composition comprising curcuminoids, acacia gum and quillaja have an improved effect in reducing LPS levels in blood, in reducing endotoxemia, in protecting the integrity of the intestinal barrier and/or in treating, decreasing and/or preventing leaky gut syndrome.

The inventors have surprisingly found that a specific composition comprising curcuminoids, acacia gum and quillaja have an improved effect in reducing intestinal lipids absorption, reducing ApoB48 levels in blood and/or in treating, decreasing and/or preventing dyslipidemia.

As shown by the examples of the present document, the composition of the invention differs from the standard turmeric extract, and allows a positive effect in reducing LPS levels in blood, in reducing endotoxemia, in protecting the integrity of the intestinal barrier and/or in treating, decreasing and/or preventing leaky gut syndrome, even using a significant lower dosage of the active compounds. Moreover, the examples of the present invention surprisingly shown that the composition of the invention reduces ApoB48 levels in blood and thus has an effect in reducing intestinal lipids absorption and dyslipidemia. Those effects were observed using a 10 to 20-fold reduction of the active daily dose of curcuminoids that is known in the art.

The reduction of the effective daily dose has the additional advantage that the administration format is reduced (i.e smaller pills can be produced) with the same efficacy. Another advantage is that the administration of the active compounds (curcuminoids) may be presented as a single dosage in a one daily dose. The presentation in smaller doses of the compositions of the invention with the possibility of one single daily dose of the active compounds will have a positive effect on liver and/or cardiovascular health.

Accordingly, in a first aspect, the invention provides a composition comprising i) curcuminoids; ii) modified starch and/or acacia gum; and iii) one or more saponins for use in reducing LPS levels in blood, in reducing endotoxemia, in protecting the integrity of the intestinal barrier and/or in treating, decreasing and/or preventing “leaky gut”.

In a second aspect, the invention provides a composition comprising i) curcuminoids; ii) modified starch and/or acacia gum; and iii) one or more saponins for use in reducing intestinal lipids absorption, reducing ApoB48 levels in blood and/or in treating, decreasing and/or preventing dyslipidemia.

In a third aspect, the invention provides the use of a composition comprising i) curcuminoids; ii) modified starch and/or acacia gum; and iii) one or more saponins for reducing LPS levels in blood, reducing endotoxemia, protecting the integrity of the intestinal barrier and/or for treating, decreasing and/or preventing “leaky gut” in a subject.

In a further aspect, the invention provides the use of a composition comprising i) curcuminoids; ii) modified starch and/or acacia gum; and iii) one or more saponins for reducing intestinal lipids absorption, reducing ApoB48 levels in blood and/or for in treating, decreasing and/or preventing dyslipidemia in a subject.

In a further aspect, the invention provides a method for maintaining both liver and/or cardiovascular health comprising the administration of an effective amount of a composition comprising i) curcuminoids; ii) modified starch and/or acacia gum; and iii) one or more saponins to a subject in need thereof.

The details, examples and preferences described herein in relation to any one of the stated aspects of the present invention will apply equally to all aspects of the present invention. Any combination of the embodiments, examples and preferences described hereinbelow in all possible variations thereof are encompassed by the present invention, unless otherwise indicated herein, or otherwise clearly contradicted by context.

### DETAILED DESCRIPTION OF THE INVENTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, etc are hereby expressly incorporated by reference for the portions of the document discussed herein, as well as in their entirety.

According to the present invention, there is provided a composition comprising i) curcuminoids; ii) modified starch and/or acacia gum; and iii) one or more saponins (such as quillaja saponins), for use in reducing LPS levels in blood, in reducing endotoxemia, in protecting the integrity of the intestinal barrier and/or in treating, decreasing and/or preventing leaky gut syndrome.

Such compositions maybe referred to hereinafter as the “composition for use of the invention” or “composition of the invention”.

Accordingly, the invention provides a composition comprising i) curcuminoids; ii) modified starch and/or acacia gum; and iii) one or more saponins (such as quillaja saponins), for use in reducing intestinal lipids absorption, reducing ApoB48 levels in blood and/or in treating, decreasing and/or preventing dyslipidemia.

The present invention, also provides the use of a composition comprising i) curcuminoids; ii) modified starch and/or acacia gum; and iii) one or more saponins (such as quillaja saponins) for reducing LPS levels in blood, reducing endotoxemia, protecting the integrity of the intestinal barrier and/or for treating, decreasing and/or preventing leaky gut syndrome in a subject.

The present invention, also provides the use of a composition comprising i) curcuminoids; ii) modified starch and/or acacia gum; and iii) one or more saponins for reducing intestinal lipids absorption, reducing ApoB48 levels in blood and/or in treating, decreasing and/or preventing dyslipidemia.

Such use maybe referred to hereinafter as the “use of the invention”.

There is also provided a method for reducing LPS levels in blood, reducing endotoxemia, protecting the integrity of the intestinal barrier and/or for treating, decreasing and/or preventing leaky gut syndrome in a subject, comprising the administration of an effective amount of a composition comprising i) curcuminoids; ii) modified starch and/or acacia gum; and iii) one or more saponins (such as quillaja saponins) to a subject in need thereof.

According to the present invention, there is also provided a method for reducing LPS levels in blood, reducing endotoxemia, protecting the integrity of the intestinal barrier and/or for treating, decreasing and/or preventing leaky gut syndrome in a subject, comprising the administration of an effective amount of a composition comprising i) curcuminoids; ii) modified starch and/or acacia gum; and iii) one or more saponins (such as quillaja saponins) to a subject in need thereof.

Such method maybe referred to hereinafter as the “method of the invention”.

In certain embodiments of the composition for use, the use, or the method of the invention, the composition comprising curcuminoids may be administered or used to provide curcuminoids in an amount of from about 50 mg/dose to about 300 mg/dose, such as about 60 mg/dose, 70 mg/dose, 80 mg/dose, 90 mg/dose, 100 mg/dose, 150 mg/dose, 200 mg/dose or 300 mg/dose.

The composition of the invention can be used to down-regulate the response to LPS and therefore to treat or prevent the occurrence of endotoxemia in a patient.

Endotoxins (Lipopolysaccharides, LPS), are a major component of the outer membrane of Gram-negative bacteria, and are known to be contributors to the inflammation [Catorce M. N., Gevorkian G. LPS-induced Murine Neuroinflammation Model: Main Features and Suitability for Pre-clinical Assessment of Nutraceuticals. Curr. Neuropharmacol. 2016; 14:155-164]. Although LPS may originate from skin and mucous membranes or local sites of bacterial infection for instance, the gut microbiota is considered the main natural reservoir of pro-inflammatory endotoxins in the body. Endotoxins (LPS) are released when bacteria die, and then dissociated endotoxins are able to cross the gastro-intestinal barrier to end up in the bloodstream. The presence of LPS in the bloodstream is defined as endotoxemia.

Briefly, the endotoxic metabolic pathway includes the binding of circulating LPS to LPS-Binding Protein (LBP) and its transfer to the CD14 receptor, which is present both in a membrane-anchored form (mCD14) and in a soluble circulating form (sCD14). The complex LPS-LBP-CD14 initiates the secretion of pro-inflammatory cytokines, such as Interleukin-6 (IL-6) or Tumor Necrosis Factor α (TNFα), through a TLR4-dependent mechanism.

Although LPS is detectable at low concentrations in the circulation of healthy individuals, there is evidence that LPS levels transiently increase following ingestion of fat-rich meals [Brown B. I. Nutritional Management of Metabolic Endotoxemia: A Clinical Review. Altern. Ther. Health Med. 2017; 23:42-54]. Such endotoxemia is defined as “metabolic endotoxemia”, in contrast to other sources of endotoxemia.

Metabolic endotoxemia has been proposed as a major cause of inflammation, including chronic low-grade inflammation. Indeed, animal and experimental studies have demonstrated that postprandial state may result in an inflammatory response closely associated with the increase in the circulating levels of LPS.

In certain embodiments, the endotoxemia is postprandial endotoxemia. In certain embodiments, the postprandial endotoxemia is after ingestion of dietary fat, such as a high saturated fat diets. In certain embodiments, the endotoxemia is postprandial endotoxemia after fat-rich meal intake.

In certain embodiments, the endotoxemia is associated with inflammatory bowel disease.

Elevated levels of LPS have been associated with a large range of diseases such as obesity and coronary artery disease (Brown B. I. Nutritional Management of Metabolic Endotoxemia: A Clinical Review. Altern. Ther. Health Med. 2017; 23:42-54).

Thus, in certain aspects, the invention relates to a method for maintaining liver and/or cardiovascular health comprising the administration of an effective amount of a composition comprising i) curcuminoids; ii) modified starch and/or acacia gum; and iii) one or more saponins to a subject in need thereof.

Thus, in certain aspects, the invention relates to a method for treating or preventing obesity and/or treating or preventing coronary artery disease comprising the administration of an effective amount of a composition comprising i) curcuminoids; ii) modified starch and/or acacia gum; and iii) one or more saponins to a subject in need thereof.

LPS may be measured using methods as described in the result part of the present text or using well stablished methods and kits such as the ones described in http://assets.thermofisher.com/TFS-Assets/BID/Reference-Materials/highly-sensitive-assay-endotoxin-detection-quantitation-variety-sample-types-white-paper.pdf.

In the present invention, ‘leaky gut syndrome’ or ‘leaky gut syndrome; LGS’ refers to a phenomenon that an intestinal mucosal cell maintains a certain gap between cells, and then a polymer material can reciprocate through the gap between the cells by applying any stimulus or damage during the process of digestion and absorption to increase an intestinal mucosa permeability. ‘Leaky gut syndrome’ collectively means a symptom caused by a phenomenon in which a polymer material in the blood leaks into the intestinal lumen due to a failure to function in the intestine properly or the polymer material in the lumen enters the blood directly. The above symptom appears in various clinical conditions such as aging, allergy, multiple trauma, rheumatoid arthritis, inflammatory large bowel disease, chronic fatigue syndrome, and irritable bowel syndrome. In addition, a pathogen, an antigen, a decay substance, or the like is introduced into the intestinal mucosa due to increased permeability of the intestinal mucosa or damage to the intestinal mucosa, causing various inflammatory reactions, and an endotoxin is introduced into a bloodstream, causing bacterial translocation and intestinal endotoxemia, which results in various inflammatory and immune responses.

The exact cause of leaky gut syndrome has not been found, but leaky gut syndrome may be caused by the use of nonsteroidal anti-inflammatory drugs (NSAIDs) as a pain reliever for a long time, the use of an antibiotic and a steroid, In the case of receiving radiation therapy or anticancer chemotherapy as anticancer therapy, in case a change in a composition of the Normal intestinal flora occurs in an intestinal track, In the case of breeding of fungi in the intestinal track, ingestion of excessive foods, ingestion of spoiled foods or ingestion of heavy metals or toxic substances, In the case of ingestion of excessively irritating foods or hypersensitivity to certain foods, excessive drinking of alcohol, multiple trauma, exposure to acute/chronic mental stress, chronic infection of bacteria, parasites and yeast in the intestinal track, and the like.

Specifically, the composition for alleviating, preventing or treating leaky gut syndrome may also be referred to as a composition having an effect of improving, preventing, alleviating, inhibiting and treating that the permeability of the intestinal track is increased due to the above various causes. The composition has the effect of alleviating, preventing, and treating leaky gut syndrome which causes various diseases as the intestinal permeability increases so that the polymeric substance in the blood leaks into the intestinal lumen or the polymeric substance in the lumen enters the branched blood. Prevention or treatment of the leaky gut syndrome may mean suppressing a phenomenon in which a gap between the intestinal mucosal cells is loosened, thus increasing the permeability of the intestinal mucosa through which the polymer substance can reciprocate.

The term “effective amount” as used herein refers to an amount of an active ingredient or a pharmaceutical composition that induces a biological or medical response to a tissue system, an animal or a human that is considered by a researcher, a veterinarian, a doctor or other clinician, and includes an amount that induces relief of the relevant symptom. The effective amount and the number of administrations for the active ingredient of the present invention may vary depending on the desired effect. Therefore, an optimal dosage to be administered can be easily determined by those skilled in the art, and be adjusted according to a variety of factors including a degree of diseases or condition to be treated (such as the degree of the symptom of the inflammatory bowel disease, a degree of intestinal permeability to the leaky gut syndrome), a content of the active ingredients and other ingredients contained in the composition, a type of the formulation, and an age, a weight, a general health condition, a sex and a diet of the patient, administration time, administration route and secretion rate of the composition, treatment period, and concomitant drugs.

Apolipoprotein B48 (ApoB48) is an apolipoprotein essential for lipid absorption in the small intestine. The dietary lipid is taken up by small intestine epithelial cells, synthesized with ApoB48 into chylomicron, and secreted into blood via lymphatic circulation. ApoB48 is a recognized characteristic marker for enterogenic lipoproteins. Research shows that in some pathological conditions, such as hyperlipidemia, coronary heart disease, insulin resistance, diabetes and other metabolic diseases, patients show abnormal fasting plasma ApoB48 level. Plasma ApoB48 levels were elevated, causing triglyceride-rich lipoprotein remnant deposition on the vessel wall, forming atherosclerotic plaques. Therefore, regulating the level of small intestine ApoB48 is helpful for improving exogenous lipid absorption, thereby reducing the risk of cardiovascular diseases caused by hyperlipidemia and the like.

The accumulation of remnant lipoproteins, especially intestine-derived chylomicron remnants (Apob48), is related to impaired lipid and glucose metabolism and ASCVD (atherosclerotic cardiovascular diseases) events.

Thus, the invention is also related to the use of a composition comprising i) curcuminoids; ii) modified starch and/or acacia gum; and iii) one or more saponins for treating, decreasing and/or preventing atherosclerotic cardiovascular diseases.

A method for treating, decreasing and/or preventing atherosclerotic cardiovascular diseases comprising the administration of an effective amount of a composition comprising i) curcuminoids; ii) modified starch and/or acacia gum; and iii) one or more saponins to a subject in need thereof.

In one embodiment the subject is a healthy subject. In one embodiment the subject is a subject with IBS.

The composition of the invention, the composition from the “use of the invention” and the composition from the “method of the invention” may be in the form of a colloidal suspension, an emulsion, or a solid, for example, in the form of a powder.

As used herein, the term “emulsion” refers to a dispersion of droplets of one liquid in another liquid in which it is not soluble or miscible.

Sometimes the terms “colloid” and “emulsion” are used interchangeably, but as used herein the term emulsion applies when a liquid phase is dispersed in another liquid. A colloid is a dispersion of particles of one substance in another substance, wherein the particles do not settle. The particles in a colloid can be any phase of matter. So, an emulsion is a type of colloid, but not all colloids are emulsions. In certain embodiments, the composition of the invention forms a colloidal suspension, of solids, like curcuminoid particles, in a liquid.

In some embodiments, the composition of the invention does not comprise fenugreek, for example, in some embodiments, the composition of the invention does not comprise fenugreek fibre (i.e. fibre obtained or obtainable from fenugreek).

The composition of the invention may comprise small amounts of polyols and/or low molecular weight sugars with preferably 1 or 2 monosaccharide units, such as less than 5% by weight of the composition or less than 2.5% by weight of the composition. Alternatively, the composition of the invention may be free of polyols and/or low molecular weight sugars such as those with 1 or 2 monosaccharide units, i.e. some compositions do not contain any polyols and/or low molecular weight sugars, such as those with 1 or 2 monosaccharide units.

The composition of the invention may comprise particles with an average diameter (or mean diameter) of from about 200 nm, 300 nm, 400 nm, 500 nm, 600 nm, 700 nm, 800 nm, 900 nm, 1,000 nm, 1,100 nm, 1,200 nm, 1,300 nm, 1,400 nm or 1,500 nm to about 9,000 nm, 8,000 nm, 7,000 nm, 6,000 nm, 5,000 nm, 4,000 nm, 3,000 nm or 2,000 nm, such as from about 1,000 nm to about 6,000 nm. The particles may also have an average diameter of from about 200 nm to about 600 nm, or from about 300 nm to about 500 nm or about 400 nm, determined by means of laser diffraction.

For example, where the composition is in the form of an emulsion or a colloid, the composition may comprise particles having an average diameter of from about 550 nm to about 700 nm and particles having an average diameter of from about 100 nm to about 250 nm giving an average diameter of about 400 nm. The average particle size is determined by means of laser diffraction (dynamic light scattering or DLS).

Where the composition is in the form of a solid, such as a powder, the composition may, for example, comprise particles having an average diameter (or mean diameter) of from about 1,000 nm to about 6,000 nm, such as from about 2,000 nm to about 4,000 nm. The average particle size is determined by means of laser diffraction (dynamic light scattering or DLS).

The particles in the composition of the invention may be in the form of micelles.

In the composition of the invention, for example where the composition is in the form of a solid, the particles may be formed using techniques known in the art, such as spray drying.

After the formation of the particles (for example, after drying, such as spray drying) the particles may be ground and/or milled (such as ball milled) to provide a more uniform size.

The size and morphology of loaded curcumin micelle may be analysed by dynamic light scattering (DLS). For example, a Mastersizer 3000 may be used. Mastersizer 3000 uses the technique of laser diffraction to measure the particle size and particle size distribution of materials. It does this by measuring the intensity of light scattered as a laser beam passes through a dispersed particulate sample.

The curcuminoids in the composition of the invention may be obtained from any source. However, it is preferred that the curcuminoids are obtained from a natural source, i.e. the curcuminoids are not synthetic, but are plant-based.

In the methods or uses described herein, the curcuminoids may be selected from the group consisting of curcumin, demethoxycurcumin, bisdemethoxycurcumin, any phase I and phase II metabolites of any one thereof, and any mixtures of two or more thereof. For example, the phase I and/or phase II metabolites may be curcumin glucuronide, curcumin sulfate, DMC glucuronide, DMC sulfate, BDMC glucuronide, BDMC sulfate, tetra-hydrocurcumin (THC), THC glucuronide, THC sulfate, hexahydrocurcumin (HHC), HHC glucuronide, HHC sulfate, or mixtures thereof.

In the composition for use, method, or use described herein, the curcuminoids may be in their unmetabolized form (i.e. free form), for example the forms of curcumin, DMC and BDMC that have not undergone glucuronide or sulfate addition.

The composition of the invention may comprise at least about 10%, at least about 25% curcuminoids, at least about 30%, at least about 40%, at least about 50%, at least about 60%, at least about 70%, at least about 80%, at least about 90%, at least about 95%, at least about 99% curcuminoids by weight of the composition, based on the total weight of the composition.

For example, in the methods or uses described herein, the curcuminoids may be present in the composition in an amount from about 20% to about 60%, such as from about 25% to about 50%, or from about 28% to about 48% by weight of the composition, based on the total weight of the composition.

For example, in the methods or uses described herein, the curcuminoids may be present in the composition in an amount from about 30 mg, 40 mg, 45 mg, 50 mg, 55 mg, 60 mg, 65 mg, 70 mg, 75 mg, 80 mg, 85 mg or 90 mg to about 1,500 mg, 1,400 mg, 1,200 mg, 1,100 mg, 1,000 mg, 900 mg, 800 mg, 700 mg, 600 mg, 500 mg, 400 mg, 300 mg, 250 mg, 200 mg, 150 mg, 100 mg or 95 mg, such as from about 70 mg to about 300 mg, or from about 70 mg to about 200 mg, or from about 70 mg to about 100 mg, such as 90 mg.

The curcuminoids may be provided by extraction of the root (rhizome) of turmeric (Curcuma longa), optionally followed by a purification step, i.e. the curcuminoids may be in the form of an extract or purified extract of turmeric. Preferably an extract or purified extract comprising from about 30% to about 100% curcuminoids, such as from about 35%, 40%, 45%, 50%, 55%, 60%, 65%, 70%, 75%, 80%, 85% or 90% to about 95%, 90%, 85%, 80%, 75%, 70%, 65%, 60%, 55%, 50% or 45% curcuminoids (wt % based on the total weight of the extract).

The turmeric may be extracted using an alcohol-based extraction solvent, such as a water/alcohol mixture or an alcohol, an organic based extraction solvent or any other technique and solvent that allows having high yields of curcuminoids. For example, the alcohol-based extraction solvent may be water/methanol (i.e. a mixture of water and methanol) or water/ethanol (i.e. a mixture of water and ethanol) or methanol or ethanol.

Where the extraction solvent comprises a water/alcohol mixture the ratio of water to alcohol may be from about 25:75 to about 1:99, such as from about 20:80 to about 5:95 or about 10:90. For example, the ex-traction solvent may be water/ethanol in a ratio of from about 25:75 to about 1:99, such as from about 20:80 to about 5:95 or about 10:90

The turmeric extract may then be further purified to provide an extract of curcuminoids comprising from about 30% to about 100% curcuminoids, such as from about 35%, 40%, 45%, 50%, 55%, 60%, 65%, 70%, 75%, 80%, 85% or 90% to about 95%, 90%, 85%, 80%, 75%, 70%, 65%, 60%, 55%, 50% or 45% curcuminoids (wt % based on the total weight of the extract).

The purification of the extract may be performed using techniques known in the art. Typically, the extract is purified using an alcohol-based solvent, such as 100% methanol or 100% ethanol.

The turmeric extract may optionally be dried to remove any excess solvent.

In certain embodiments of the composition for use, use, or method of the invention, the curcuminoids are provided by extraction and optionally purification from the root (rhizome) of turmeric (Curcuma longa), oleoresin turmeric root, defatted oleoresin turmeric root and mixtures thereof, such as ethanol extraction.

Where the curcuminoids are provided in the form of a turmeric extract as previously defined, the composition may comprise from about from about 30% to about 80% turmeric extract, such as from about 35% to about 45% by weight of the composition. For example, the composition may comprise from about 35% (i.e. 35%) to about 45% turmeric extract by weight of the composition, where the turmeric extract comprises from about 85% to about 95% curcuminoids by weight of the turmeric extract, providing a composition that comprises from about 30% (i.e. 30%) to about 43% curcuminoids by weight of the composition.

The curcuminoids may be provided as a liquid or a powder, preferably as a powder. For example, a powdered turmeric extract.

As used herein, the term “curcuminoids” includes curcumin, demethoxycurcumin (DMC), and bisdemethoxycurcumin (BDMC). For example, the turmeric extract may comprise from about 70% to about 85% curcumin (such as from about 75% to about 80%), from about 10% to about 25% DMC (such as from about 15% to about 20%) and from about 0% to about 10% BDMC. For example, the composition may comprise from about 60 mg to about 90 mg of curcuminoids, with from about 54 mg to about 69 mg curcumin and from 6 mg to about 11 mg of DMC and BDMC. In a preferred embodiment, the composition may be administered or used to provide curcuminoids in an amount of from about 70, 80, 90, 95, 100, 150, 200, 250 mg/day to about 1,500, 1,400, 1,300, 1,200, 1,100, 1,000, 900, 850, 800, 750, 700, 650, 600, 550, 500, 450, 400, 350, 300 mg/day of curcuminoids, such as from about 70 mg/day to about 150 mg/day of curcuminoids, such as from about 70 mg/day to about 90 mg/day of curcuminoids. In an embodiment the ratio between curcumin and DMC/BDMC is from 7:3 to 8:2. In a preferred embodiment, the curcumin provided is from about 54 mg/day to about 69 mg/day and the DMC and BDMC is from 16 mg/day to about 21 mg/day.

In the methods or uses described herein, the acacia gum (also referred to as Arabic gum) in the composition may be present in an amount from about 30%, 35%, 40%, 45%, 50%, 55%, to about 85%, 80%, 75%, 70%, 65%, 60% by weight of the composition, such as from about 50% to about 60% by weight of the composition or about 58% by weight of the composition.

In the methods or uses described herein, the modified starch in the composition may be present in an amount from about 40% to about 65% by weight of the composition, such as from about 50 to about 60% by weight of the composition or about 58% by weight of the composition.

Saponins are a group of naturally occurring glycosides, predominantly found in plants. Saponins comprise a non-carbohydrate aglycone coupled to sugar chain units. Saponins are divided in two groups: steroidal and triterpene saponins. Over 100 steroidal and an even higher number of triterpene saponins have been so far identified. (K. Hostettmann, & A. Marston, Saponins (Cambridge University Press 1995). As used herein, the term “saponin(s)” includes one or more saponins from natural (such as quillaja or yucca saponins) or synthetic origin as well as an extract obtained or obtainable from any vegetal, animal or bacterial source of saponin, for example quillaja, yucca etc. The saponin in the composition of the present invention can be of natural origin or of synthetic origin. It may be one or more saponins from the same or different origin. For example, the saponin(s) can be obtained from plants such as soya, beans, peas, Solanum and Allium species, tomato, asparagus, tea, peanut, spinach, sugar beet, yam, blackberry, liquorice root, primula root, senega root, tea, ginseng, Quillaja (such as Quillaja saponaria), Yucca (such as Yucca shidigera), and/or Gyposphila. In one embodiment the one or more saponins is quillaja saponin(s). In one preferred embodiment, the saponin is not a ginger saponin.

The one or more saponins used in the present invention can be highly purified or may be a natural extract with different concentrations of saponins.

In certain embodiments, the one or more saponin(s) is selected from purified quillaja saponin(s) from natural or synthetic origin or an extract obtained or obtainable from quillaja and mixtures thereof.

As used herein, the term “Quillaja saponin(s)”, “Yucca saponi(s)”, etc, means one or more saponins that can be obtained or be obtainable from any of the members of the quillaja family, or the Yucca family or any of the plants that contains saponins such as the ones described before. The Quillaja saponin or the mixture of Quillaja saponins (or the yucca saponin or the mixture of Yucca saponins) can be of synthetic or natural origin.

As will be appreciated by the person skilled in the art, as used herein the term “obtainable from” means that the saponin(s) may be obtained from a plant or may be isolated from the plant, or may be obtained from an alternative source, for example by chemical synthesis or enzymatic production. Whereas the term “obtained” as used herein, means that the saponin(s) is directly derived from the plant. For example, in one embodiment, the saponin(s) can be a “natural extract comprising saponin(s)”.

The at least one saponin, may be of natural or synthetic origin.

A “purified saponin(s)” means one or more saponins of natural or synthetic origin that have a concentration of at least about 80%, at least about 90%, at least about 95%, at least about 99%, at least about 99.9% of one or more saponins as described before (such as Quillaja saponin(s) and/or Yucca saponin(s)).

A “saponin(s) comprising extract” means any natural extract comprising at least one type of saponin as described before that may be derived from, e.g., but not limited to soya, beans, peas, oat, Solanum and Allium species, tomato, asparagus, tea, peanut, spinach, sugar beet, yam, blackberry, liquorice root, primula root, senega root, Quillaja (such as Quillaja saponaria), Yucca (such as Yucca shidigera), and/or Gyposphila.

According to the present invention, the at least one saponin may be derived from a single source or from multiple sources.

According to the present invention, the at least one saponin comprising extract may be derived from a single source or from multiple sources.

In one embodiment the saponin component is a natural extract, such as a Quillaja extract, tea extract, licorice extract, beet root extract, sugar beet extract, ginseng extract, oat extract, Yucca extract or a mixture thereof, with at least 5% wt/wt, or at least 10% wt/wt, or at least 15% wt/wt, or at least 20% wt/wt, or at least 25% wt/wt, or at least 30% wt/wt, or at least 35% wt/wt, or at least 40% wt/wt, or at least 50% wt/wt, or at least 60% wt/wt, or at least 70% wt/wt, or at least 80% wt/wt of saponins. In one embodiment the saponin component may be a Quillaja extract with at least 60% saponins, such as 65% wt/wt of saponins.

In a preferred embodiment, the saponin(s) is Quillaja. As used herein, the term “Quillaja” includes one or more purified Quillaja saponins from natural or synthetic origin as well as an extract obtained or obtainable from Quillaja.

The saponin(s) (such as Quillaja saponins) of the composition of the invention may be present in an amount from about 0.1%, 0.2%, 0.3%, 0.4%, 0.5%, 0.6%, 0.7%, 0.8%, 1%, 1.2%, 1.3%, 1.4%, 1.5%, 1.6%, 1.7%, 1.8%, 1.8%, 2%, 2.5%, 3%, 3.5%, 4% or 4.5% to about 5%, 4.5%, 4%, 3.5%, 3%, 2.5%, 2.4%, 2.3%, 2.2%, 2%, 1.5%, 1%, 0.8%, 0.6%, 0.5%, or to 0.2% by weight of the composition, such as from about 0.4% to about 3%, such as from 0.5% to about 2.5% or about 0.65% or about 2% by weight of the composition. In a preferred embodiment, the one or more saponin(s) are one or more Quillaja saponin(s). In one embodiment, the Quillaja saponins are purified Quillaja saponins. In a more preferred embodiment, the one or more saponins may be provided as an extract obtained or obtainable from Quillaja.

In the present invention “purified Quillaja saponins” may refer to one of more Quillaja saponins from natural or synthetic origin that have a purity of at least 80%, such as at least 90%, such as at least 95%, such as at least 99%.

The saponin(s) (such as a purified Quillaja saponin(s) or such as Quillaja extracts from or obtainable from Quillaja containing saponin(s)) used in the process of the invention may be in any form, such as a liquid or a solid. For example, the saponin(s) may be used in the form of a solid, such as a powder.

The Quillaja saponin(s) (such as a purified Quillaja saponins or the extract obtained from or obtainable from Quillaja containing saponin(s)) used in the process of the invention may be in any form, such as a liquid or a solid. For example, the Quillaja extract may be used in the form of a solid, such as a powder.

When present in the composition of the invention, the saponin(s) (such as Quillaja saponins) water and/or other solvent, such as alcohol, may be added to the solid or liquid saponin(s) (such as Quillaja).

In a preferred embodiment, the one or more saponins are Quillaja saponin(s) and are present in the final composition from about 0.5% to about 5%, such as about 0.8 to about 2%, such as from about 1% to 1.5% such as about 1.3% wt/wt.

In the methods or uses described herein, the composition may optionally comprise a vegetable oil. For example, the composition of the invention may comprise vegetable oils selected from the group consisting of coconut oil, corn oil, cottonseed oil, olive oil, palm oil, peanut oil (ground nut oil), rapeseed oil, including canola oil, safflower oil, sesame oil, soybean oil, sunflower oil, and mixtures thereof.

The vegetable oil present in the composition of the invention may be present in an amount of from about 1% to about 20% vegetable oil, such as from about 2.5% to about 10% or about 5% by weight of the composition.

In a preferred embodiment of the composition for use, method or use described herein, the composition comprises from about 30 to about 40% of Curcuma longa rhizome ethanol extract (with at least 80%, such as at least 90% of curcuminoids), from about 55 to about 65% acacia gum, from about 3 to about 7%, sunflower oil and from about 1 to about 3% Quillaja extract.

Unless otherwise stated herein, the weight percentages listed are based on the total weight of (dry) composition obtained.

In the method or use described herein, the composition comprising curcuminoids may be provided in the form of a (suitable) composition, such as a “pharmaceutical composition” or a “food composition”.

In particular embodiments, the composition comprising curcuminoids may be provided in the form of a pharmaceutical composition (which may also be referred to as a pharmaceutical formulation or a veterinary composition) or functional food composition comprising curcuminoids and optionally a pharmaceutically acceptable excipient or (functional) food acceptable ingredient, as appropriate.

A “functional food composition” as used herein refers to a nutraceutical composition, a functional food composition, a dietary or food product for humans or animals (such as functional food compositions, i.e. food, drink, feed or pet food or a food, drink, feed or pet food supplements) or a nutritional supplement. Functional food composition can be presented as beverages, dairy products, bakery products, etc.

When used in a functional food, such as a beverage, a dairy product, a bakery product, the composition of the invention is incorporated into said food product (for example in a liquid or solid form) so as to provide the effective amounts of curcuminoids (such as at least 90 mg of curcuminoids).

As used herein, references to pharmaceutically (or veterinary) acceptable excipients may refer to pharmaceutically (or veterinary) acceptable adjuvants, diluents and/or carriers as known to those skilled in the art.

Food acceptable ingredients include those known in the art (including those also referred to herein as pharmaceutically acceptable excipients) and that can be natural or non-natural, i.e. their structure may occur in nature or not. In certain instances, they can originate from natural compounds and be later modified (e.g. maltodextrin).

By “pharmaceutically acceptable” (or veterinary acceptable) we mean that the additional components of the composition are sterile and pyrogen free. Such components must be “acceptable” in the sense of being compatible with the extract of the invention and not deleterious to the recipients thereof. Thus, “pharmaceutically acceptable” includes any compound(s) used in forming a part of the formulation that is intended to act merely as an excipient, i.e. not intended to have biological activity itself. Thus, the pharmaceutically acceptable excipient is generally safe, non-toxic, and neither biologically nor otherwise undesirable. The skilled person will understand that extracts of the invention (e.g. in the form of compositions, such as pharmaceutical compositions, as known to those skilled in the art, such as those as described herein) may be administered to a patient or subject (e.g. a human or animal patient or subject) by any suitable route, such as by the oral, rectal, nasal, pulmonary, buccal, sublingual, transdermal, intracisternal, intraperitoneal, and parenteral (including subcutaneous, intramuscular, intrathecal, intravenous and intradermal) route. In particular, extracts of the invention may be administered orally. In such instances, pharmaceutical compositions according to the present invention may be specifically formulated for administration by the oral route. Suitable pharmaceutical (or veterinary) carriers include inert solid diluents or fillers, sterile aqueous solutions and various organic solvents. Examples of solid carriers are lactose, terra alba, sucrose, cyclodextrin, maltodextrin, talc, gelatin, silica, agar, pectin, acacia, magnesium stearate, stearic acid, arabic gum, modified starch and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Pharmaceutical (or veterinary) compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art. Liquid dosage forms for oral administration include solutions, emulsions, aqueous or oily suspensions, syrups and elixirs.

Compositions (e.g. pharmaceutical or food compositions) described herein, such as those intended for oral administration, may be prepared according to methods known to those skilled in the art, such as by bringing the components of the composition into admixture.

Such compositions as described herein may contain one or more additional components selected from the group consisting of food ingredients, such as sweetening agents, flavouring agents, colouring agents and preserving agents. Tablets may contain the active ingredient(s) in admixture with non-toxic pharmaceutically acceptable excipients (or ingredients) which are suitable for the manufacture of tablets. These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In a preferred embodiment the composition is formulated to provide a single dose of curcuminoids in an amount of from about 50 mg/dose, about 60 mg/dose, 70 mg/dose, 80 mg/dose, 90 mg/dose, 100 mg/dose, 150 mg/dose, 200 mg/dose 300 mg/dose, to about 1,400 mg/dose, 1,300 mg/dose, 1,200 mg/dose, 1,100 mg/dose, 1,000 mg/dose, 900 mg/dose, 800 mg/dose, 700 mg/dose, 600 mg/dose, 500 mg/dose, 400 mg/dose, such as from about 70 mg/dose to about 100 mg/dose, such as about 90 mg/dose.

In a preferred embodiment of the methods/uses described herein, the composition may be formulated and/or administrated as a single dose formulation. For example, the composition may be formulated as a single dose (for example a 300 mg formulation) with a curcuminoids contain of at least 90 mg.

Moreover, because of the very high efficacy of the composition of the invention, the dosage of curcuminoids (such as the daily dose) can be also reduced.

In the methods/uses described herein, the composition may be administered or used to provide curcuminoids in an amount of from about 50 mg/day, about 60 mg/day, 70 mg/day, 80 mg/day, 90 mg/day, 100 mg/day, 150 mg/day, 200 mg/day, 300 mg/day, to about 1,400 mg/day, 1,300 mg/day, 1,200 mg/day, 1,100 mg/day, 1,000 mg/day, 900 mg/day, 800 mg/day, 700 mg/day, 600 mg/day, 500 mg/day, 400 mg/day, such as from about 70 mg/day to about 100 mg/day, such as about 90 mg/day. For example, the composition may be formulated as a single dose (for example of 300 mg) with a curcuminoids contain of at least 90 mg/day.

For example, the composition may provide curcuminoids in an amount of from about 1 to about 10 mg/kg of body weight, such as from about 2.5 to about 7.5 mg/kg of body weight, such as about 5 mg/kg, or such as about 1.29 mg/kg (90 mg curcuminoids for 70 kg of body weight).

In a preferred embodiment, the dosage is of 300 mg and the daily dose of curcuminoids is from about 70 mg/day to about 150 mg/day of curcuminoids, such as from about 70 mg/day to about 100 mg/day of curcuminoids, such as about 90 mg/day of curcuminoids.

The timing and duration of administration of the composition of the invention can vary. For instance the composition may be administered or used before a fat containing food.

In certain embodiments of the composition for use, the use, or method of the invention, the composition may be administered or used at least 2 days before a fat containing food is eaten by or administer to the subject, at least one day before the fat containing food is eaten or administered, or some hours before the fat containing food and optionally also administrated one day after the fat containing food.

Fat containing food can be considering as a food that comprises at least 10%, at least 20%, at least 30% w/w of fat.

In an embodiment of the invention, the method of delivering is orally in liquid, solid or paste forms. Solid dosage forms for oral administration may include capsules, tablets, caplets, pills, troches, chewables, lozenges, powders, and granules. A capsule typically comprises a core material comprising a composition of the invention and a shell that encapsulates the core material. The core material may be solid, liquid, or an emulsion. The shell material may comprise soft gelatin, hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Some such polymers may also function as taste-masking agents.

Tablets, pills, and the like may be compressed, multiply compressed, multiply layered, and/or coated. The coating may be single or multiple. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextran, maltodextrin, cyclodextrins, inulin, pectin, mannans, gum arabic, locust bean gum, mesquite gum, guar gum, gum karaya, gum ghatti, tragacanth gum, funori, carrageenan, agar, alginates, chitosan, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. Tablets and pills may additionally be prepared with enteric coatings.

For example, if the final product is a beverage, the composition of the invention will be added to the final product to obtain a final dose of curcuminoids of from about 70, 80, 90, 100, 150, 200, 250 mg/day to about 1,000, 900, 850, 800, 750, 700, 650, 600, 550, 500, 450, 400, 350, 300 mg/day of curcuminoids, such as from about 70 mg/day to about 150 mg/day of curcuminoids, such as from about 70 mg/day to about 90 mg/day of curcuminoids. For example, the composition may provide curcuminoids in an amount of from about 1 to about 10 mg/kg of body weight, such as from about 2.5 to about 7.5 mg/kg of body weight or about 5 mg/kg, such as about 1.29 mg/kg (90 mg curcuminoids for 70 kg of body weight).

Alternatively, powders or granules embodying the compositions disclosed herein may be incorporated into a food product. In a preferred embodiment, the food product may be a drink for oral administration. Non-limiting examples of a suitable drink include fruit juice, a fruit drink, an artificially flavored drink, an artificially sweetened drink, a carbonated beverage, a sports drink, a liquid diary product, a shake, and so forth to which excipients may also be added. Other suitable means for oral administration include aqueous and nonaqueous solutions, emulsions, suspensions and solutions and/or suspensions reconstituted from non-effervescent granules, containing suitable solvents, preservatives, emulsifying agents, suspending agents, diluents, sweeteners, coloring agents, and flavoring agents. Additionally, chewable forms, as disclosed in U.S. Pat. No. 7,223,417, incorporated herein wholly by reference, may be used.

The beverage products disclosed here optionally contain a flavor composition, i.e., one or more flavor components, for example, natural or synthetic fruit flavors, botanical flavors, other flavors, and mixtures of any of them. As used here, the term “fruit flavor” refers generally to those flavors derived from the edible reproductive part of a seed plant. Included are both those wherein a sweet pulp is associated with the seed, e.g., banana, tomato, cranberry and the like, and those having a small, fleshy berry. The term berry also is used here to include aggregate fruits, i.e., not “true” berries, but those that are commonly accepted as a berry. Also included within the term “fruit flavor” are synthetically prepared flavors made to simulate fruit flavors derived from natural sources. Examples of suitable fruit or berry sources include whole berries or portions thereof, berry juice, berry juice concentrates, berry purees and blends thereof, dried berry powders, dried berry juice powders, and the like.

The composition may be a solid foodstuff. Suitable examples of a solid foodstuff include a food bar, gummies, a snack bar, a cookie, a brownie, a muffin, a cracker, a chewable gum, an ice cream bar, a frozen yogurt bar, a chewy snack and the like. In an embodiment, the method comprises administering the composition as described above multiple times per day, or if taken as a liquid, may be mixed and taken throughout the day.

In an alternative or further embodiment of a method of delivery, the composition may be used in conjunction with a fat containing food. For example, the composition may be given before, during, or immediately after the ingestion of a fat containing food.

Typically the period of administration of the composition of the invention comprising curcuminoids (such as in a pharmaceutical or food composition comprising curcuminoids) in the uses of methods of the invention described herein, is of more than 2 days, more than 3 days more than 4 days, more than 5 days, more than 6 days, more than 7 days; more than 1 week, more than 2 weeks, more than 3 weeks, more than 4 weeks, more than 5 weeks, more than 6 weeks, more than 7 weeks, more than 8 weeks, more than 9 weeks, more than 10 weeks, more than 1 month, more than 1 months, more than 2 months, more than 3 months, more than 4 months, more than 5 months, more than 6 months, more than 7 months, more than 8 months, more than 9 months, more than 10 months, more than 11 months, more than 12 months.

“Subject” refers to a human or an animal, including, but not limited to, mice, rats, rabbits, dogs, cats, pigs, cows, and non-human primates, including, but not limited to, monkeys and chimpanzees. In a preferred embodiment the subject is a human (female or male).

“Administration” or “administering” refers to routes of introducing a compound or composition provided herein to an individual to perform its intended function. An example of a route of administration that can be used includes, but is not limited to oral, parenteral administration, such as subcutaneous, intravenous, or intramuscular injection or infusion, etc.

“Healthy subject” refers to an individual who is not known to suffer of any significant illness and corresponds to the general population.

As used herein, the term “treatment” (and, similarly, “treating”) takes its normal meaning in the field of medicine. In particular, the term may refer to achieving a reduction (for example, at least a 10% reduction, such as at least a 20%, 30% or 40% reduction, e.g. at least a 50% reduction) in the severity of one or more clinical symptom associated with the disease or disorder (e.g. endotoxemia or leaky gut syndrome), as may be determined using techniques known to those skilled in the art (for example, by a medical physician) or the ones described in the examples and/or to slowing the progression of the disease or disorder (i.e. increasing the amount of time taken for the disease or disorder to progress to a more severe state, e.g. when compared to the time expected to be taken in a patent not so treated). As used herein, the term “prevention” (and, similarly, “preventing”) includes references to the prophylaxis of the disease or disorder (and vice-versa). In particular, the term may refer to achieving a reduction in the likelihood of the patient (or healthy subject) developing the condition (for example, at least a 10% reduction, such as at least a 20%, 30% or 40% reduction, e.g. at least a 50% reduction).

As used herein in relation to medical conditions, the term “reducing” may refer to making the observed quantity smaller or decrease in size (i.e. reducing LPS levels in blood). In particular, the term may refer to achieving a reduction in the likelihood of the patient (or healthy subject) developing the condition (for example, at least a 10% reduction, such as at least a 20%, 30% or 40% reduction, e.g. at least a 50% reduction).

To define the reduction of LPS levels in blood or the reduction of ApoB48 levels in blood, after the amount or concentration of the LPS levels are defined, said value may be compared to a cut-off value as established in a reference population. The expression “comparing the value of LPS . . . to the cut-off value as established in a reference population” is merely used to further illustrate what is obvious to the skilled artisan anyway.

In one embodiment of the invention, the control value is established using a control sample, which is obtained from an individual or preferably from a control population of individuals (also referred to as reference population) known to be free of a given condition, i.e. low LPS or low Apo48 levels in blood. Alternatively, the control value is defined based on the analysis of a population of individuals known to have a certain condition (high levels of LPS or high levels of ApoB48 in blood). Here the control value has to be chosen in order to be below the values typically determined for this population. Finally, the control value may be obtained by assessing a healthy population (low values of LPS or ApoB48) and a diseased population (high values of LPS or Apo48) and established a cut-off value in order to define the % of reduction of said values (LPS or Apo48 in blood) if compared with the control group. It is within the skills of the practitioner to choose an appropriate control sample/population and/or a control value for the markers established therein.

It will be appreciated by the skilled artisan that the absolute marker values established in a control will be dependent on the assay used. Preferably samples from 100 or more well-characterized individuals from the appropriate control/reference population are used to establish a control value. Also preferred the control/reference population may be chosen to consist of at least 20, 30, 50, 200, 500 or 1000 individuals. Healthy individuals represent a preferred reference population for establishing a control value.

The treating or reduction achieved by the compositions of the invention, may be obtained after 24h or ingestion, or after 48h, after 72h, after 4 days, after 5 days, after 1 week or the consumption of the composition of the invention.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

The term “about” as used herein, e.g. when referring to a measurable value (such as an amount or weight of a particular component in the reaction mixture), refers to variations of ±20%, ±10%, ±5%, ±1%, ±0.5%, or, particularly, ±0.1% relative to the specified amount. For example, a variation of ±0.5% with regards to the percentage of a component in the composition of the invention, means a variation of 0.5% relative to the percentage given, i.e. ±0.5% of 10% would mean a variation from 9.5% to 10.5%.

### FIGURES

FIG. 1. LPS blood translocation. Blood levels of LPS before and 5 hours after fat challenge (time 0). While no main treatment effect on post-prandial response was observed for LPS, a trend for a baseline effect of the Test Formulation according to the invention (Supplement A) on LPS levels (p=0.06) was found if compared with the control (Supplement B).

FIG. 2. Chylomicron production. ApoB48 is a chylomicron-specific biomarker of chylomicron, which incorporate LPS and lipids for their blood translocation, A significant effect of the Test Formulation according to the invention (Supplement A) was found on postprandial ApoB48 levels (p<0.001) and ApoB48 AUC (p=0.04) compared with the control (Supplement B).

FIG. 3. Study design.

### EXAMPLES

### 1. Test Formulation

A Test Formulation (TPG) was composed of: natural powdered extract obtained from turmeric (Curcuma longa) root, acacia gum, sunflower oil and Quillaja extract. It was developed and was intended for use as a food supplement. It has already been tested for safety in pre-clinical and human studies.

The Test Formulation comprised: 30-40% of Curcuma longa rhizome ethanol extract, 55-65% acacia gum, 3-7%, sunflower oil, 1-3% Quillaja extract. The Quillaja component was an extract powder containing approximately 65% saponins by dry weight. The curcuminoids content was 30% w/w (23% (w/w) curcumin and 7% (w/w) DMC and BDMC (% w/w in respect to total weight of the Test Formulation). The Test Formulation was prepared in the form of capsules.

The Test Formulation was selected for in vivo study on fat-induced intestinal barrier disruption as measured with LPS translocation in IBS patients with a diarrhea-predominant subtype (IBS-D). The primary objective of this study was to determine the effect of short-term Test Formulation (Turmipure GOLD®) supplementation on LPS translocation in IBS-D patients after a high-fat challenge. The secondary objective of this study was to determine the effect of short term Test Formulation (Turmipure GOLD®) supplementation on gastrointestinal complaints and LPS-related biomarkers in IBS-D patients after a high-fat challenge.

In this double-blind, randomized, placebo-controlled cross-over trial 20 adult (18-70 yrs) IBS-D patients were included.

The participants ingested three times a single dose (300 mg) of turmeric or placebo.

The primary study parameter is blood LPS levels. The secondary study parameters are blood levels of LPS-related biomarkers.

Blood sampling was performed via a cannula.

### 2. Study Design

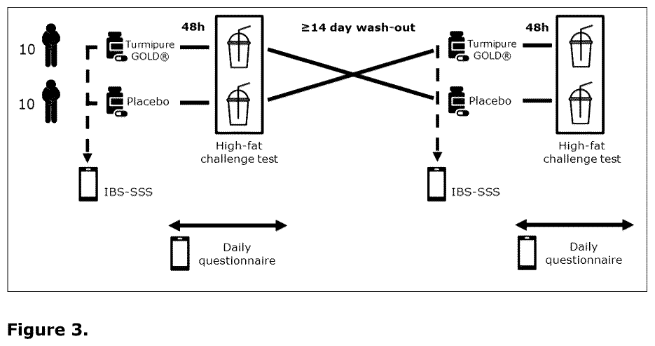

To study the effect of the Test Formulation on postprandial LPS translocation. LPS-related biomarkers, and gastrointestinal complaints in IBS-D patients, a double-blind, randomized, placebo-controlled cross-over trial was conducted (FIG. 3).

Before the start of supplementation (day −3), participants filled out a questionnaire on the severity of their IBS-related gastrointestinal complaints (IBS-SSS). Next, participants consumed the Test Formulation capsules (300 mg) or placebo (Dyed Acacia Gum) at three occasions: day −2 and −1, and on the morning of the high-fat challenge test days. They furthermore consumed a standardized meal the evening before the test day with a high-fat challenge test. The two test days were separated by at least 14 days wash-out. On the test day, participants consumed a high-fat shake followed by 5 hourly blood collections and they filled out short questionnaires on gastrointestinal complaints.

### 2.1 Population (Base)

The study included 20 adult IBS-D patients. This subgroup of IBS patients was chosen as it had been shown that they had in general increased plasma LPS levels, indicating reduced intestinal barrier function and low grade inflammation. They furthermore indicated more often to have gastrointestinal complaints following consumption of high-fat foods. The intervention could therefore be especially beneficial for this subgroup. As increased BMI is also known to have an effect on intestinal barrier function and low grade inflammation it was chosen to apply a somewhat higher upper limit in BMI range for the inclusion criteria.

### 2.2 Inclusion Criteria

In order to be eligible to participate in this study, a subject had to meet all of the following criteria:
IBS patients that meet the Rome IV criteria+additional criteria specific for the diarrhea-predominant subtype, based on the most frequent self-reported stool types using the Bristol stool chart (BSC).
Male and female adults, aged 18-70 years;
Having a Body Mass Index (BMI) between 18.5 and 30 kg/m2;
Willing to keep a stable dietary pattern throughout the study.

### 3. Investigational Product/Treatment

Participants were supplemented with 1 capsule per day of Test Formulation and placebo during 3 days: 48 hour before the high-fat challenge test, 24 hour before the high-fat challenge test and on the day of the high-fat challenge test. Capsules containing 300 mg were consumed with water before breakfast (between 7.00-8.00 hr).

### 4. Main Study Parameter/Endpoint

The main study parameter was LPS translocation measured as levels of LPS in venous blood samples collected at baseline and 5 hourly after high-fat shake consumption.
LPS was determined by Pyrochrome-LAL kit (Associates of Cape Cod Inc).

### 4.1. Secondary Study Parameters/Endpoints

The secondary parameters were gastrointestinal complaints and LPS-related biomarkers in venous blood samples collected at baseline and 5 hourly after high-fat shake consumption.
Gastrointestinal complaints were measured via repeated short questionnaires (at T=1, 2, 3, 4, and 5 hour)
abdominal pain, bloating, flatulence, nausea, heartburn
LPS-related biomarkers:
   ApoB48 concentration was assessed using a commercial sandwich ELISA method (Fujifilm Wako Shibayagi Corp).
   LBP was measured by commercially available ELISA assays (Sanbio BV)
   sCD14 was measured by commercially available ELISA assays (Sanbio BV)

All laboratory analyses were conducted in the laboratory facilities of Wageningen Food & Biobased Research.

### 4.2. Randomisation, Blinding and Treatment Allocation

Study participants were randomized over the two cross-over arms (Test Formulation→Placebo and Placebo→Test Formulation). Blocked randomization for BMI and age was applied. To ensure similar BMI and age in both treatment groups, participants were sorted by BMI and age, and for each block of two participants the two possible treatment orders (1→2 and 2→1) were randomly assigned.

Any other clinically relevant differences in baseline characteristics were accounted for in the statistical analysis (covariance analysis).

Both capsules were uniformly packaged, but with a coded label that specifically corresponded to the intervention, to ensure double-blinding. After analyses of all data, product coding was unblinded for the investigators.

Baseline IBS complaints and daily questionnaire

At day −3, subjects filled out the online IBS-SSS questionnaire.

At day −3 to day +2, subjects filled out a short daily questionnaire via an app (LifeData, LLC), asking amongst others for GI complaints and when applicable supplement compliance.

### Supplement Intake

Subjects ingested one capsule (with water) in the morning before breakfast, between 7:00 h and 8:00 h (supplement or placebo), of day −2, day −1 and at the research unit in the morning of the test day. After at least 14 days washout and cross-over, subjects ingested again one capsule (with water) in the morning before breakfast, between 7:00 h and 8:00 h (supplement or placebo), of day −2, day −1 and at the research unit in the morning of the second test day.

### Test Day (‘Day 0’)

Subjects consumed a standardised low fat evening meal prior to the test days and were asked to refrain from alcohol and strenuous exercise. After an overnight fast, all subjects came to the research facility by own transport. The subjects consumed the third and last supplement capsule (at T=−1 hour). A study nurse inserted a cannula to allow blood collection throughout the day. Thereafter, a baseline (t=0) blood sample was collected. After the baseline measurements, the participant consumed the high-fat shake within 10 minutes (T=0). Postprandial blood samples were collected hourly for 5 hours from the cannula after consumption of the shake. At each collection it was sampled 11 mL of blood through the cannula (4 mL EDTA plasma, 4 mL serum, 3 mL dummy to flush cannula) resulting in a total blood volume collection of 66 mL per test day.

Statistical analyses were done using SPSS statistics. A p-value of p<0.05 is considered as statistically significant. In general, the continuous data is presented as mean±standard deviation (SD). Data may be log-transformed to normalize data distribution before parametric statistical analyses.

### Results.

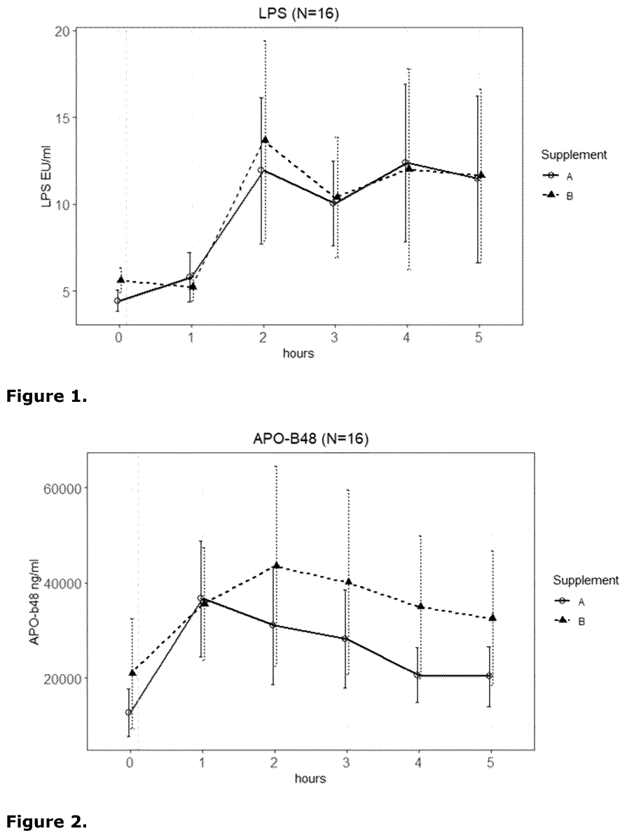

As it can be seen on FIG. 1, a trend for a baseline effect of the Test Formulation (Supplement A) on LPS levels (p=0.06) was found.

As it can be seen on FIG. 2. A significant effect of Test Formulation (Supplement A) was found on postprandial ApoB48 levels (p<0.001) with lower concentrations of ApoB48 observed 2 hours, 3 hours, 4 hours and 5 hours after the high-fat challenge test. A significantly lower Area under the concentration-time curve (ApoB48 AUC) was also observed (p=0.04).

1. (canceled)

2. (canceled)

3. (canceled)

4. (canceled)

5. A method for maintaining liver health, cardiovascular health, reducing LPS levels in blood, in reducing endotoxemia, in protecting the integrity of the intestinal barrier, in treating, decreasing and/or preventing leaky gut syndrome, in reducing intestinal lipids absorption, in reducing ApoB48 levels in blood, and/or in treating, decreasing and/or preventing dyslipidemia:
comprising administering an effective amount of a composition comprising i) curcuminoids; ii) modified starch and/or acacia gum; and iii) one or more saponins to a subject in need thereof.

6. The method according to claim 5, wherein the composition comprising curcuminoids is administered to provide curcuminoids in an amount selected from the group consisting of from about 50 mg/dose to about 300 mg/dose, about 60 mg/dose, 70 mg/dose, 80 mg/dose, 90 mg/dose, 100 mg/dose, 150 mg/dose, 200 mg/dose and 300 mg/dose.

7. The method according to claim 5, wherein the curcuminoids are curcumin and its phase I or phase II metabolites, demethoxycurcumin and its phase I or phase II metabolites, bisdemethoxycurcumin and its phase I or phase II metabolites and mixtures thereof.

8. Composition for use, use, of The method according to claim 7, wherein curcuminoids are selected from curcumin, demethoxycurcumin (DMC), bisdemethoxycurcumin (BDMC), and mixtures thereof.

9. The method according to claim 5, wherein the composition comprises an amount of curcuminoids selected from the group consisting of 30 mg, 40 mg, 45 mg, 50 mg, 55 mg, 60 mg, 65 mg, 70 mg, 75 mg, 80 mg, 85 mg, or from about 90 mg to about 1500 mg, 1400 mg, 1200 mg, 1110 mg, 1000 mg, 900 mg, 800 mg, 700 mg, 600 mg, 500 mg, 400 mg, 300 mg, 250 mg, 200 mg, 150 mg, 100 mg or 95 mg, from about 70 mg to about 300 mg, from about 70 mg to about 200 mg, from about 70 mg to about 100 mg, such as and 90 mg.

10. The method according to claim 5, wherein the acacia gum is in an amount from about 30%, 35%, 40%, 45%, 50%, 55%, to about 85%, 80%, 75%, 70%, 65%, 60% by weight of the composition, based on the total weight on the composition.

11. The method according to claim 5, wherein the one or more saponins is selected from Quillaja saponins, yucca saponins, tea saponins, peanut saponins, spinach saponins, sugar beet saponins, yam saponins, blackberry saponins, liquorice root saponins, primula root saponins, ginseng saponins, and mixtures thereof.

12. The method according to claim 5, wherein the at least one saponin(s) is present in an amount selected from the group consisting of about 0.1% to about 5% by weight of the composition, from about 0.5% to about 3% by weight of the composition, and from about 0.5% to about 1.3% by weight of the composition, based on the total weight on the composition.

13. The method according to claim 5, wherein the composition comprises particles having an average diameter selected from the group consisting of from about 100 nm to about 10,000, nm from about 100 nm to about 700 nm, and from about 1,000 nm to about 6,000 nm measured using laser diffraction.

14. The method according to claim 5, wherein the composition is administered to provide curcuminoids in an amount selected from the group consisting of from about 50 mg/day, about 60 mg/day, about 70 mg/day, about 80 mg/day, about 90 mg/day, about 100 mg/day, about 150 mg/day, about 200 mg/day, about 300 mg/day, to about 1400 mg/day, about 1,300 mg/day, about 1,200 mg/day, about 1,100 mg/day, about 1,000 mg/day, about 900 mg/day, about 800 mg/day, about 700 mg/day, about 600 mg/day, about 500 mg/day, and about 400 mg/day.

15. The method according to claim 5, wherein the composition is administered or dosed in a dosage of from about 100 mg to about 500 mg, and wherein the daily dose of curcuminoids is from about 50 mg/day to about 500 mg/day.

16. The method according to claim 15 wherein the composition is administered to provide curcuminoids in an amount of about 70 mg/day to about 90 mg/day of curcuminoids, with from about 54 mg/day to about 69 mg/day curcumin and from 16 mg/day to about 21 mg/day of DMC and BDMC.

17. The method according to claim 5, wherein the composition is administered as a single daily dose.

18. The method according to claim 5, wherein the composition is administered to before a fat containing food is eaten by the subject.

19. The method according to claim 5, wherein the composition is administered at least 2 days before a fat containing food is eaten by the subject, optionally is also administrated one day after the fat containing food has been eaten by the subject.

20. The method according to claim 5, wherein the composition comprises a pharmaceutical, a nutraceutical or a food composition.

21. The method according to claim 5, wherein the subject is a human.

22. The method according to claim 5, wherein the subject suffers from IBS.

23. The method according to claim 10, wherein the acacia gum is in an amount from about 50% to about 60% by weight of the composition, based on the total weight on the composition.

24. The method according to claim 14, wherein the composition is administered to provide curcuminoids in an amount of from about 70 mg/day to about 100 mg/day.

25. The method according to claim 5, wherein the composition is administered or dosed in a dosage of about 300 mg, and wherein the daily dose of curcuminoids is from about 70 mg/day to about 90 mg/day.
